(19) United States
(12) Patent Application Publication
(10) Pub. No.: US 2026/0096763 A1
(43) Pub. Date: Apr. 9, 2026

(54) BIOSIGNAL MEASUREMENT SYSTEM

(71) Applicant: NTT, Inc., Tokyo (JP)

(72) Inventors: Kenichi Matsunaga, Tokyo (JP); Kento Watanabe, Tokyo (JP)

(21) Appl. No.: 19/107,896

(22) PCT Filed: Sep. 1, 2022
(86) PCT No.: PCT/JP2022/032932

§ 371 Date: Feb. 28, 2025

(51) Int. Cl. A61B 5/28; A61B 5/00; A61B 5/308; A61B 5/332

(52) U.S. Cl. CPC A61B 5/28; A61B 5/0024; A61B 5/0028; A61B 5/308; A61B 5/332; A61B 5/6824; A61B 2560/0214

(57) ABSTRACT

An embodiment is a biosignal measurement system including two electrode devices and a biosignal generation device. Each electrode device has an electrode for measuring a biopotential in a target human body, an amplifier circuit for amplifying the measured biopotential, a frequency modulation (FM) transmitter for converting an output signal from the amplifier circuit into an FM signal, an FM receiver for receiving an FM signal from the other electrode device and converting it into a voltage signal, an adjustment circuit for adjusting the voltage signal from the FM receiver and providing it as an input to the amplifier circuit, and a wireless transmitter for transmitting biopotential information. The biosignal generation device receives the biopotential information from both electrode devices and generates a biosignal waveform based on the received biopotential information.

### CROSS-REFERENCE TO RELATED APPLICATIONS

This application is a national phase entry of PCT Application No.

PCT/JP2022/032932, filed on Sep. 1, 2022, which application is hereby incorporated herein by reference.

### TECHNICAL FIELD

The present invention relates to a biosignal measurement system.

### BACKGROUND

In electrocardiogram measurement, which is one of biopotential measurements, a potential difference between electrodes disposed on both left and right sides of the human body is measured. For example, as illustrated in FIG. 6, there has been proposed a measurement system in which a device 301 is attached to a central portion of a body, and an electrode 304 is provided to contact with left and right waist portions by causing a wire 303 to extend along a compression wear 302 (Non Patent Literature 1).

### CITATION LIST

### Non Patent Literature

Non Patent Literature 1: Nahoko Kasai and others, “Development of Functional Textile ”hitoe“: Wearable Electrodes for Monitoring Human Vital Signals” Communication Society Magazine, 2017, Vol. 11, No. 1, pp.17-23, The Institute of Electronics, Information and Communication Engineers.

### SUMMARY

### Technical Problem

However, attachment on the body causes a feeling of discomfort due to a feeling of pressure and a feeling of avoidance due to taking labor for attachment. Other than the body, for example, it is conceivable to attachment to four limbs. However, in this case, since the wire connecting the left and right electrodes forms a loop like handcuffs, it strongly restricts the movement of the body. These problems can be solved as long as the wire between the left and right electrodes can be cut and the devices can be separated into two units, but the reference of potential measurement cannot be determined through the separation, and it becomes difficult to perform the biopotential measurement.

Embodiments of the present invention has been made to solve the above-described problems, and an object thereof is to enable a biopotential to be easily measured even when the wire between two electrodes is cut and the devices are separated into two units.

### Solution to Problem

According to embodiments of the present invention, there is provided a biosignal measurement system including two electrode devices and a biosignal generation device, in which each of the two electrode devices includes: an electrode that measures a biopotential in a target human body; a non-inverting amplification circuit that inputs the measured biopotential to a non-inverting amplifier terminal, amplifies the biopotential, and outputs the amplified biopotential from an output terminal; a quantization circuit that converts an amplified signal output from the output terminal of the non-inverting amplifier circuit into digital data and generates biopotential information; a wireless transmitter that transmits the biopotential information to the biosignal generation device; an FM transmitter that converts a voltage signal output from the output terminal of the non-inverting amplifier circuit into an FM signal and transmits the FM signal to the other electrode device; an FM receiver that receives the FM signal transmitted from the other electrode device to an own electrode device, converts the FM signal into a voltage signal, and outputs the voltage signal; an adjustment circuit that outputs the voltage signal output from the FM receiver to an inverting input terminal of the non-inverting amplifier circuit as an adjustment signal adjusted under a set condition; and a power supply that supplies power to the non-inverting amplifier circuit, the quantization circuit, the wireless transmitter, the FM transmitter, the FM receiver, and the adjustment circuit, a frequency of the FM signal transmitted from one of the two electrode devices to the other electrode device and a frequency of the FM signal transmitted from the other electrode device to the one electrode device have different frequencies, and the biosignal generation device includes a wireless receiver that receives the biopotential information transmitted from each of the two electrode devices, and an arithmetic circuit that generates a biosignal waveform by using the biopotential information received by the wireless receiver.

### Advantageous Effects

As described above, according to embodiments of the present invention, two electrode devices and the biosignal generation device are connected by wireless communication, and the two electrode devices are connected by FM communication. Therefore, even when the wire between the two electrodes is cut and the devices are separated into two units, the biopotential can be easily measured.

### BRIEF DESCRIPTION OF THE DRAWINGS

FIG. 1A is a configuration diagram illustrating a configuration of a biosignal measurement system according to a first embodiment of the present invention.

FIG. 1B is a configuration diagram illustrating a partial configuration of a biosignal measurement system according to the first embodiment of the present invention.

FIG. 2 is an explanatory diagram illustrating a concept of a biosignal measurement system according to the first embodiment of the present invention.

FIG. 3 is a configuration diagram illustrating a configuration of a biosignal measurement system according to a second embodiment of the present invention.

FIG. 4 is a configuration diagram illustrating a configuration of a biosignal measurement system according to a third embodiment of the present invention.

FIG. 5 is a configuration diagram illustrating a configuration of a biosignal measurement system according to a fourth embodiment of the present invention.

FIG. 6 is a configuration diagram illustrating a configuration of a biosignal measurement system of the related art.

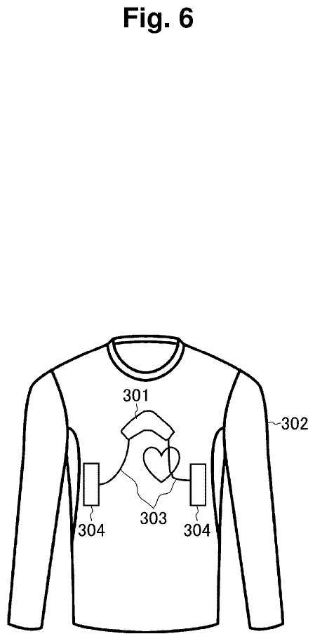

### DETAILED DESCRIPTION OF ILLUSTRATIVE EMBODIMENTS

A biosignal measurement system according to an embodiment of the present invention will be described below.

### First Embodiment

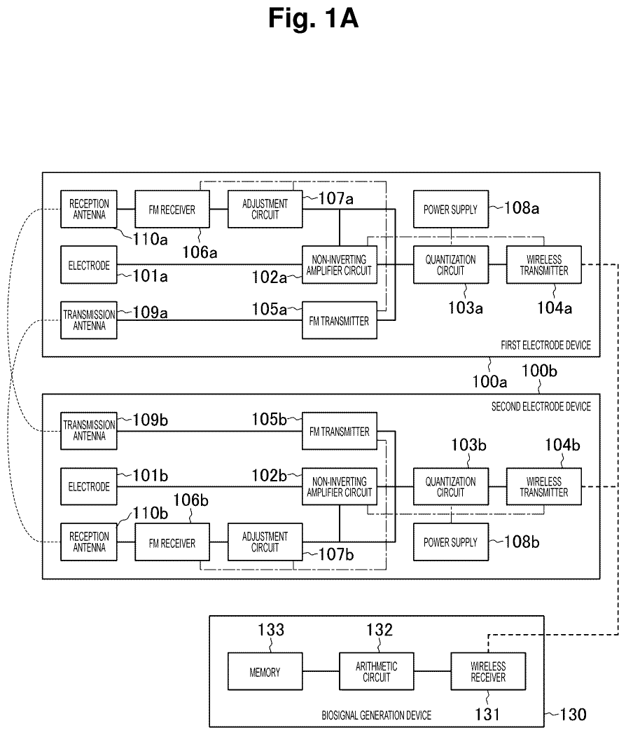

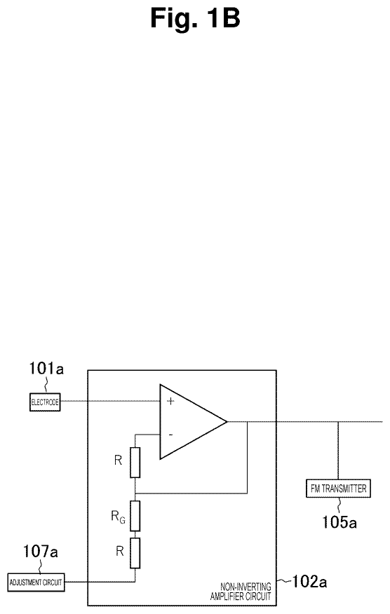

First, a biosignal measurement system according to a first embodiment of the present invention will be described with reference to FIGS. 1A and 1B. This system includes two of a first electrode device 100a and a second electrode device 100b, and a biosignal generation device 130.

First, the first electrode device 100a includes an electrode 101a that measures a biopotential in a target human body, a non-inverting amplifier circuit 102a that inputs the measured biopotential to a non-inverting amplifier terminal, amplifies the biopotential, and outputs the amplified biopotential from an output terminal, a quantization circuit 103a that converts the amplified signal output from the output terminal of the non-inverting amplifier circuit 102a into digital data to generate biopotential information, and a wireless transmitter 104a that transmits the biopotential information to the biosignal generation device 130.

First, the first electrode device 100a includes an FM transmitter 105a, an FM receiver 106a, and an adjustment circuit 107a. The FM transmitter 105a converts a voltage signal output from the output terminal of the non-inverting amplifier circuit 102a into an FM signal, and transmits the converted FM signal to the second electrode device 100b through a transmission antenna 109a. The FM receiver 106a converts the FM signal transmitted from the second electrode device 100b to the first electrode device 100a and received through a reception antenna 110a into a voltage signal and outputs the voltage signal. In the first electrode device 100a, the adjustment circuit 107a outputs the voltage signal output from the FM receiver 106a as an adjustment signal adjusted in a set condition to an inverting input terminal of the non-inverting amplifier circuit 102a.

The output of the non-inverting amplifier circuit 102a is also input to the inverting input terminal. For example, as illustrated in FIG. 1B, when a signal input from the adjustment circuit 107a to the inverting input terminal of the non-inverting amplifier circuit 102a is Vdev2 and an output of the non-inverting amplifier circuit 102a is Vout1 at a negative terminal input Vin− of an operational amplifier of the non-inverting amplifier circuit 102a, the signals are mixed at a ratio of “Vin−=(R+RG)/(2R+RG)Vout+(R)/(2R+RG)Vdev2” and input to the inverting input terminal of the non-inverting amplifier circuit 102a. The same applies to a non-inverting amplifier circuit 102b to be described later.

Furthermore, the first electrode device 100a includes a power supply 108a that supplies power to the non-inverting amplifier circuit 102a, the quantization circuit 103a, the wireless transmitter 104a, the FM transmitter 105a, the FM receiver 106a, and the adjustment circuit 107a.

First, the second electrode device 100b includes an electrode 101b that measures a biopotential in a target human body, the non-inverting amplifier circuit 102b that inputs the measured biopotential to a non-inverting amplifier terminal, amplifies the biopotential, and outputs the amplified biopotential from an output terminal, a quantization circuit 103b that converts the amplified signal output from the output terminal of the non-inverting amplifier circuit 102b into digital data to generate biopotential information, and a wireless transmitter 104b that transmits the biopotential information to the biosignal generation device 130.

Furthermore, the second electrode device 100b includes an FM transmitter 105b, an FM receiver 106b, and an adjustment circuit 107b. The FM transmitter 105b converts a voltage signal output from the output terminal of the non-inverting amplifier circuit 102b into an FM signal, and transmits the converted FM signal to the first electrode device 100a through a transmission antenna 109b. The FM receiver 106b converts the FM signal transmitted from the first electrode device 100a to the second electrode device 100b and received through a reception antenna 110b into a voltage signal and outputs the voltage signal. The adjustment circuit 107b outputs the voltage signal output from the FM receiver 106b as an adjustment signal adjusted in a set condition to an inverting input terminal of the non-inverting amplifier circuit 102b.

Furthermore, the second electrode device 100b includes a power supply 108b that supplies power to the non-inverting amplifier circuit 102b, the quantization circuit 103b, the wireless transmitter 104b, the FM transmitter 105b, the FM receiver 106b, and the adjustment circuit 107b.

Here, the frequency of the FM signal transmitted from the first electrode device 100a to the second electrode device 100b is different from the frequency of the FM signal transmitted from the second electrode device 100b to the first electrode device 100a.

The biosignal generation device 130 includes a wireless receiver 131 that receives the biopotential information transmitted from each of the first electrode device 100a and the second electrode device 100b, and an arithmetic circuit 132 that generates a biosignal waveform by using the biopotential information received by the wireless receiver 131. For example, the arithmetic circuit 132 can generate an electrocardiogramal waveform by using two pieces of biopotential information transmitted from each of the first electrode device 100a and the second electrode device 100b, which are attached to any two positions of four limbs of the human body. Furthermore, the biosignal generation device 130 includes a memory 133 that stores the biosignal waveform generated by the arithmetic circuit 132.

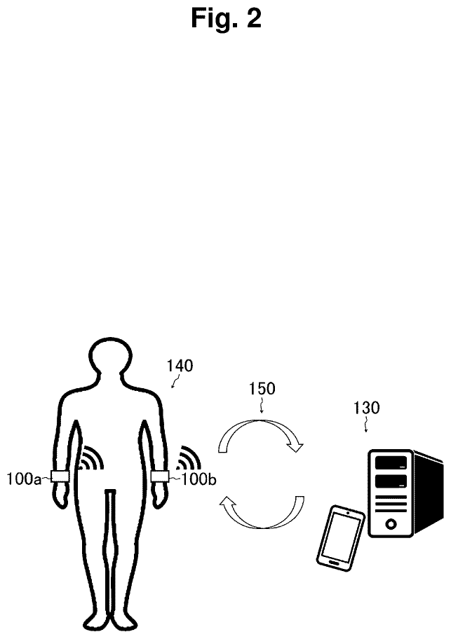

FIG. 2 illustrates a concept of the biosignal measurement system according to the first embodiment. For example, in a case where a cardiac potential signal is measured as a biosignal to generate an electrocardiogram, it is necessary to dispose a plurality of electrodes on a positional relationship so as to position the heart between the electrodes. Therefore, for example, it is conceivable to attach the first electrode device 100a and the second electrode device 100b to at least two positions of four limbs such as arms and legs as a measurement site to be easily used in a human body 140. By adopting such an attachment form of the first electrode device 100a and the second electrode device 100b, it is possible to greatly reduce a sense of pressure or discomfort caused by wearing a wear or the like. Note that the measurement target of the biosignal measurement system is not limited to the cardiac potential signal, and the biosignal measurement system can be applied to measure a myogenic potential signal, a brain wave, and the like. When the biosignal measurement system is applied, discomfort of the wire is eliminated, a degree of freedom of electrode arrangement is increased, and an effect of widening a width of a device to be mounted is also expected.

In this example, the non-inverting amplifier circuit 102a of the first electrode device 100a and the non-inverting amplifier circuit 102b of the second electrode device 100b are coupled to each other. Since such a configuration is similar to the configuration of an oscillation circuit, oscillation occurs when the phase rotation is 180 degrees and the amplification degree is one or more in the phase rotation and amplification degree caused by the delay of the signals to be coupled with each other.

As an example, in a case where a non-inverting amplifier circuit typically having a bandwidth of DC to 1 kHz in the biosignal is built, when a delay of 0.1 ms occurs, the phase rotation of 36 degrees occurs in a 1 kHz signal. That is, since the phase rotation of 180 degrees occurs with a delay of 0.5 ms, the possibility of oscillation cannot be denied. Therefore, in the mutual coupling between the non-inverting amplifier circuit 102a and the non-inverting amplifier circuit 102b described above, it is necessary to minimize the delay at a portion to which the voltage signal is coupled.

Next, the electrode 101a and the electrode 101b will be described. As these electrodes, various electrodes can be used, and any electrodes such as an Ag/AgCl electrode used also in medical application, a conductive cloth electrode, and a metal electrode can be used. In particular, it is also possible to further improve the usability by adopting a non-contact electrode configuration in which a sensor device is attached on the clothes by using a cloth or metal electrode that does not need to be adhered to the human body. In particular, for the non-contact electrode configuration, capacitive coupling is suitable since it is easy to pass through in high frequency communication.

Next, the adjustment circuit 107a and the adjustment circuit 107b will be described. These are portions that perform adjustment by a constant multiplication on the basis of the received FM signal, and thus can be configured by the operational amplifier. Although the operational amplifiers can be connected in multiple stages, the delay is accumulated every time the operational amplifiers are connected in multiple stages, and thus the operational amplifiers are likely to be unstable. Therefore, it is preferable that each of the adjustment circuit 107a and the adjustment circuit 107b comprises a minimum one-stage operational amplifier.

Next, the non-inverting amplifier circuit 102a and the non-inverting amplifier circuit 102b will be described. Since the biopotential is a very weak signal, signal amplification by the non-inverting amplifier circuit 102a and the non-inverting amplifier circuit 102b each configured by a filter circuit and an amplifier circuit configured by an operational amplifier is required. By adopting the non-inverting amplifier circuit, it is possible to realize a configuration equivalent to that of an instrumentation amplifier having a high common mode suppression capability as a system.

Furthermore, in the amplification stages of the non-inverting amplifier circuit 102a and the non-inverting amplifier circuit 102b, a high input impedance is required in order to reduce the loss of the biopotential, but even when the non-inverting amplifier circuit 102a and the non-inverting amplifier circuit 102b have a high input impedance configuration, the noise hardly increases. On the other hand, with an inverting amplifier circuit, the resistance for determining the input impedance also affects the gain setting, and further directly contributes as thermal noise, so that the SN ratio is lowered. Therefore, the non-inverting amplifier circuit is effective.

Since the potential difference between the two electrodes is detected in the biopotential measurement, the same reference potential is required in the non-inverting amplifier circuit 102a and the non-inverting amplifier circuit 102b. Therefore, by using the potentials generated by the adjustment circuits 107a and the adjustment circuit 107b, balanced signal amplification between the first electrode devices 100a and the second electrode device 100b is possible, and good biosignal information is finally obtained.

Furthermore, the FM communication frequency used when the non-inverting amplifier circuit 102a and the non-inverting amplifier circuit 102b are coupled to each other needs to have different frequencies. This is because when the same frequency is used, mutual interference occurs and desired coupling cannot be obtained. This corresponds to dividing a band in communication, and by increasing the frequency to be used, embodiments of the present invention can be used not only in a configuration in which electrode devices are paired, but also among more electrode devices.

Next, the wireless transmitter 104a and the wireless transmitter 104b will be described. For example, the wireless transmitter 104a may be only required to be configured by one communication module, and may be only required to be connected so as to be capable of receiving a measurement potential output from the quantization circuit 103a and transmitting the measurement potential to the biosignal generation device 130.

As a standard of a wireless communication network 150 among the wireless transmitter 104a, the wireless transmitter 104b, and the wireless receiver 131, any standards such as carrier communication, Wi-Fi (registered trademark), and Bluetooth (registered trademark) can be applied (FIG. 2). It is necessary to select a transmitter and a receiver according to a communication standard. In a short-range communication standard such as Bluetooth, a smartphone or the like which is a terminal close to a user, which is a human body to be measured, can be used as the biosignal generation device 130. Furthermore, when Wi-Fi or the like is used, a server or the like can be used as the biosignal generation device 130.

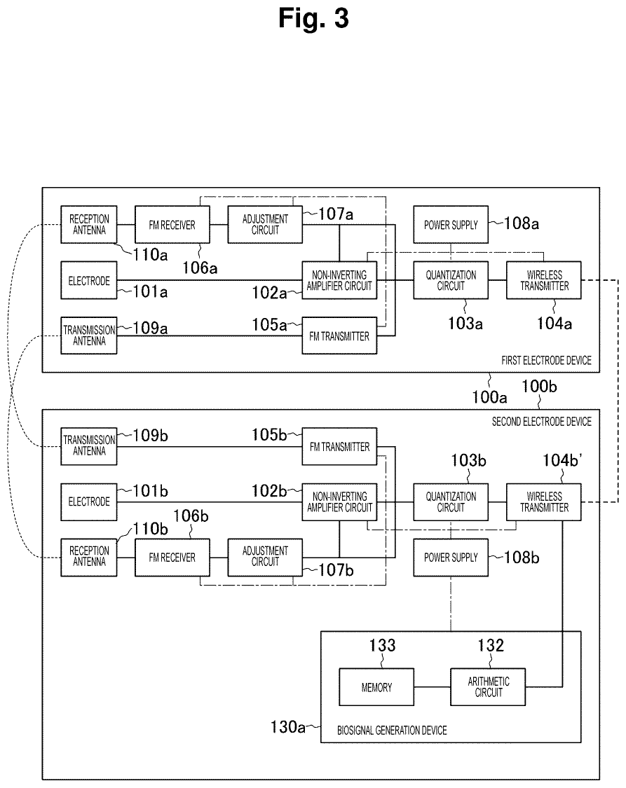

Furthermore, functions required by the biosignal generation device 130 are to receive signals from a plurality of electrode devices and to obtain a target biopotential by calculation. These functions can be implemented (incorporated) in any electrode device without using the biosignal generation device 130 (FIG. 3). In this case, as illustrated in FIG. 3, the second electrode device 100b includes a wireless receiver 104b′, and a biosignal generation device 130a including the arithmetic circuit 132 and the memory 133 is added.

The wireless receiver 104b′ of the second electrode device 100b receives the biopotential information transmitted from the first electrode device 100a, and obtains the biopotential by calculating the biopotential information together with the biopotential information from the second electrode device 100b. By storing the obtained biopotential in the memory 133 of the second electrode device 100b, the same function and effect as described above can be achieved. In addition, in this configuration, since it is not necessary to separately provide the biosignal generation device 130, it is not necessary to carry the smartphone or the like, and it is possible to implement measurement without limitation for the user.

### Second Embodiment

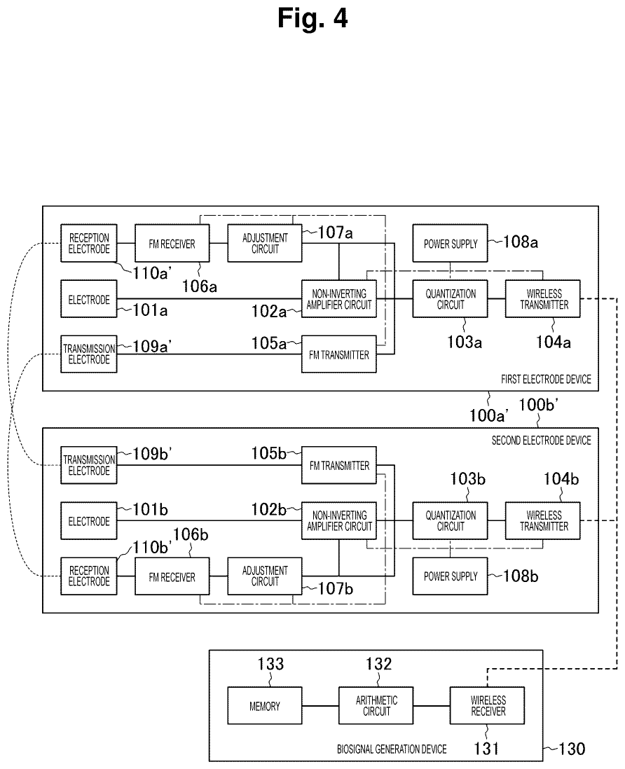

Next, a biosignal measurement system according to a second embodiment of the present invention will be described with reference to FIG. 4. This system includes two of a first electrode device 100a′ and a second electrode device 100b′, and a biosignal generation device 130.

The first electrode device 100a′ includes an electrode 101a, a non-inverting amplifier circuit 102a, a quantization circuit 103a, a wireless transmitter 104a, an FM transmitter 105a, an FM receiver 106a, an adjustment circuit 107a, and a power supply 108a. Furthermore, the second electrode device 100b′ includes an electrode 101b, a non-inverting amplifier circuit 102b, a quantization circuit 103b, a wireless transmitter 104b, an FM transmitter 105b, an FM receiver 106b, an adjustment circuit 107b, and a power supply 108b. Furthermore, the biosignal generation device 130 includes a wireless receiver 131, an arithmetic circuit 132, and a memory 133. These components are the same as those of the first embodiment described above.

In the second embodiment, FM communication is performed using a human body as a communication channel. The FM transmitter 105a converts a voltage signal output from the output terminal of the non-inverting amplifier circuit 102a into an FM signal, and transmits the converted FM signal to the second electrode device 100b′ by using a human body as a channel through a transmission electrode 109a'. The FM receiver 106a converts the FM signal transmitted from the second electrode device 100b′ to the first electrode device 100a′ by using the human body as the channel and received through a reception electrode 110a′ into a voltage signal and outputs the voltage signal.

Furthermore, the FM transmitter 105b converts a voltage signal output from the output terminal of the non-inverting amplifier circuit 102b into an FM signal, and transmits the converted FM signal to the first electrode device 100a′ by using the human body as the channel through a transmission electrode 109b′. The FM receiver 106b converts the FM signal transmitted from the first electrode device 100a′ to the second electrode device 100b′ by using the human body as the channel and received through a reception electrode 110b′ into a voltage signal and outputs the voltage signal.

As described above, by performing FM communication via the human body, delay and power are reduced, and the human body functions as a waveguide. Therefore, there is an advantage that radio waves can be confined, interference from the outside is strong, and a risk of causing interference to the outside can be reduced. Also in a case where the human body is transmitted, the frequency of the FM signal transmitted from the first electrode device 100a′ to the second electrode device 100b′ is different from the frequency of the FM signal transmitted from the second electrode device 100b′ to the first electrode device 100a′. By setting the frequency band to be used to about several MHz to 100 MHz on the basis of the electrical properties of the human body, less loss can be achieved.

Furthermore, in this example, for example, in the first electrode device 100a′, three of the electrode 101a, the transmission electrode 109a′, and the reception electrode 110a′ are used, but since the frequencies are different from each other, it is possible to provide one electrode by providing a band-pass filter, and there is an effect of improving the comfort of the user since the portion to be brought into contact with the human body is reduced.

### Third Embodiment

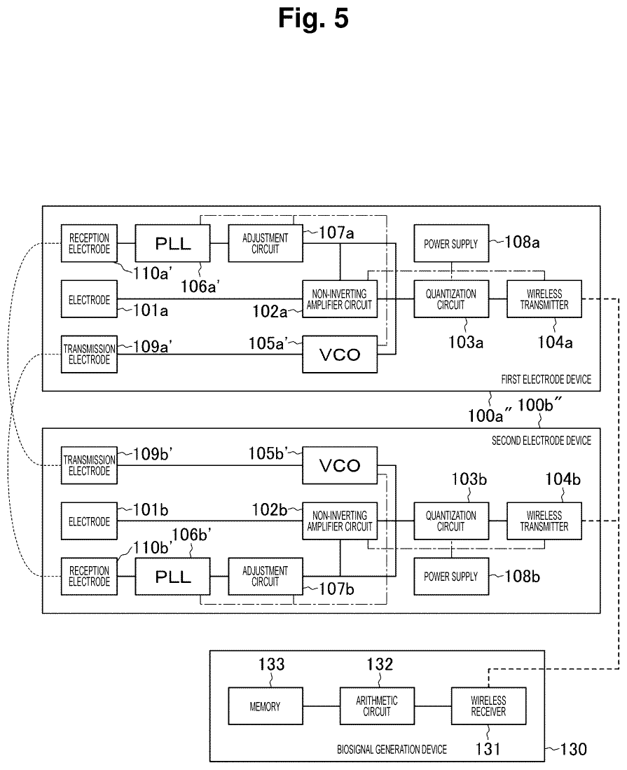

Next, a biosignal measurement system according to a third embodiment of the present invention will be described with reference to FIG. 5. This system includes two of a first electrode device 100a″ and a second electrode device 100b″, and a biosignal generation device 130.

In the third embodiment, as the FM transmitter, a voltage control oscillator (VCO 105a′, VCO 105b′) is used, and as the FM receiver, a phase locked loop (PLL 106a′, PLL 106b′) is used. The other components are the same as those of the second embodiment described above.

As described in the second embodiment, in the configuration in which the FM communication is implemented using the human body as the communication channel, there is a possibility that a parameter related to the delay greatly varies. Therefore, it is necessary to use a device having a small delay particularly at the time of transmission and reception in the FM communication, and as an example of this, first, the FM transmitter comprises the voltage control oscillator, and the output frequency is directly modulated by the voltage. Furthermore, the FM receiver comprises the phase locked loop, and set as a direct detection system.

In a case where the human body is used as a communication channel (communication path), a high SN can be expected due to a radio wave confinement effect.

Therefore, in a case where the human body is used as the channel, it is effective to employ a configuration with less delay than demodulation with high accuracy. The delay can be effectively reduced by configuring the FM transmitter as the voltage control oscillator and the FM receiver as the phase locked loop.

By the way, since the FM transmitter comprises the voltage control oscillator, and the FM receiver comprises the phase locked loop, the delay can be reduced. However, in a case where direct detection and direct modulation are adopted, it is essential for communication with less error that the voltage-to-frequency conversion characteristics match between the voltage control oscillator constituting the FM transmitter and the voltage control oscillator included in the phase locked loop constituting the FM receiver. However, the voltage control oscillator uses LC resonance caused by a variable capacitance diode generally called a varactor or oscillation caused by a ring oscillator. Due to manufacturing variations of these elements, the voltage-to-frequency conversion characteristics may not match between the voltage control oscillator constituting the FM transmitter and the voltage control oscillator included in the phase locked loop constituting the FM receiver.

As described above, as an example in which the voltage-frequency conversion characteristics do not match, in a case where the center frequencies do not match, it is possible to perform adjustment by adding an offset to the operational amplifier included in the adjustment circuit so that the voltage-frequency characteristics between the voltage control oscillator constituting the FM transmitter of the other electrode device and the voltage control oscillator of the phase locked loop of the own electrode device match. For example, the offset can be determined on the basis of the input voltage to the operational amplifier. In this manner, in a case where the FM communication is adopted, it is possible to perform adjustment without increasing the delay.

Furthermore, as an example in which the voltage-frequency conversion characteristics do not match, in a case where the slopes of the voltage-frequency characteristics do not match, the oscillation frequency characteristics are monitored, and it is possible to perform adjustment without increasing the delay by adjusting an amplification condition of the operational amplifier included in the adjustment circuit so that the voltage-frequency characteristics between the voltage control oscillator constituting the FM transmitter of the other electrode device and the voltage control oscillator of the phase locked loop of the own electrode device match. For example, the amplification condition of the operational amplifier can be adjusted by making the resistance value of the operational amplifier variable.

As described above, by appropriately adjusting the condition of the operational amplifier in the adjustment circuit, it is possible to cover the weak point in the FM communication, which is caused by the voltage control oscillator and the phase locked loop. In particular, in a case where the human body is used as the communication channel, the above-described configuration is suitable.

As described above, according to embodiments of the present invention, two electrode devices and the biosignal generation device are connected by wireless communication, and the two electrode devices are connected by FM communication. Therefore, even when the wire between the two electrodes is cut and the devices are separated into two units, the biopotential can be easily measured.

Note that the present invention is not limited to the above-described embodiments, and it is apparent to those skilled in the art that many modifications and combinations can be made within the technical idea of the present invention.

### REFERENCE SIGNS LIST

100a First electrode device
100b Second electrode device
101a, 101b Electrode
102a, 102b Non-inverting amplifier circuit
103a, 103b Quantization circuit
104a, 104b Wireless transmitter
105a, 105b FM transmitter
106a, 106b FM receiver
107a, 107b Adjustment circuit
108a, 108bPower supply
109a, 109b Transmission antenna
110a, 110b Reception antenna
130 Biosignal generation device
131 Wireless receiver
132 Arithmetic circuit
133 Memory

1-6. (canceled)

7. A biosignal measurement system comprising two electrode devices and a biosignal generation device,
wherein each of the two electrode devices includes:
an electrode for measuring a biopotential in a target human body;
an amplification circuit including a non-inverting input terminal, an inverting input terminal and an output terminal, the amplification circuit configured to amplify the biopotential received via the electrode by the non-inverting input terminal, and output an amplified signal from the output terminal;
a quantization circuit configured to convert the amplified signal output from the output terminal of the amplifier circuit into digital data to generate biopotential information;
a wireless transmitter for transmitting the biopotential information to the biosignal generation device;
an FM transmitter configured to convert the amplified signal output from the output terminal of the amplifier circuit into an FM signal and transmit the FM signal to the other electrode device;
an FM receiver configured to receive another FM signal transmitted from the other electrode device, convert the other FM signal into a voltage signal, and output the voltage signal;
an adjustment circuit configured to adjust the voltage signal output from the FM receiver under a set condition and output the voltage signal adjusted under the set condition to the inverting input terminal of the amplifier circuit as an adjustment signal; and
a power supply for supplying power to the amplifier circuit, the quantization circuit, the wireless transmitter, the FM transmitter, the FM receiver, and the adjustment circuit, and
the biosignal generation device includes
a wireless receiver configured to receive the biopotential information transmitted from each of the two electrode devices, and
an arithmetic circuit configured to generate a biosignal waveform by using the biopotential information received by the wireless receiver.

8. The biosignal measurement system according to claim 7,
wherein an FM communication between the FM transmitters and the FM receivers is provided using the human body as a communication channel.

9. The biosignal measurement system according to claim 7,
wherein one of the two electrode devices incorporates the biosignal generation device.

10. The biosignal measurement system according to claim 7,
wherein the arithmetic circuit generates an electrocardiogramal waveform by using two pieces of the biopotential information transmitted from each of the two electrode devices attached to any two positions of four limbs of the human body.

11. The biosignal measurement system according to claim 7,
wherein the FM transmitter comprises a voltage control oscillator, and
the FM receiver comprises a phase locked loop.

12. The biosignal measurement system according to claim 11,
wherein the adjustment circuit includes an operational amplifier, and is configured perform at least one of adjustment of an amplification condition of the operational amplifier and adding offset to the operational amplifier so that a voltage-frequency characteristic of the voltage control oscillator of the other electrode device and a voltage-frequency characteristic of a voltage control oscillator of the phase locked loop of the electrode device match with each other.

13. The biosignal measurement system according to claim 7,
wherein a frequency of the FM signal transmitted from one of the two electrode devices to the other electrode device and a frequency of the FM signal transmitted from the other electrode device to the one electrode device have different frequencies.

14. An electrode device comprising for a biosignal measurement system comprising two electrode devices and a biosignal generation device,
an electrode for measuring a biopotential in a target human body;
an amplification circuit including a non-inverting input terminal, an inverting input terminal and an output terminal, and configured to amplify the biopotential received via the electrode by the non-inverting input terminal, and output an amplified signal from the output terminal;
a quantization circuit configured to convert the amplified signal output from the output terminal of the amplifier circuit into digital data to generate biopotential information;
a wireless transmitter for transmitting the biopotential information;
an FM transmitter configured to convert the amplified signal output from the output terminal of the amplifier circuit into an FM signal and transmit the FM signal to another electrode device;
an FM receiver configured to receive another FM signal transmitted from the other electrode device, convert the other FM signal into a voltage signal, and output the voltage signal;
an adjustment circuit configured to adjust the voltage signal output from the FM receiver under a set condition and output the voltage signal adjusted under the set condition to the inverting input terminal of the amplifier circuit as an adjustment signal; and
a power supply for supplying power to the amplifier circuit, the quantization circuit, the wireless transmitter, the FM transmitter, the FM receiver, and the adjustment circuit.

15. The electrode device according to claim 14,
wherein the FM transmitter comprises a voltage control oscillator, and
the FM receiver comprises a phase locked loop.

16. The electrode device according to claim 15,
wherein the adjustment circuit includes an operational amplifier, and is configured perform at least one of adjustment of an amplification condition of the operational amplifier and adding offset to the operational amplifier so that a voltage-frequency characteristic of the voltage control oscillator of the other electrode device and a voltage-frequency characteristic of a voltage control oscillator of the phase locked loop of the electrode device match with each other.

17. A biosignal measurement system comprising:
a first electrode device and a second electrode device, each electrode device comprising:
an electrode configured to measure a biopotential in a target human body;
an amplifier circuit configured to amplify the measured biopotential;
a frequency modulation (FM) transmitter configured to convert an output signal from the amplifier circuit into an FM signal;
an FM receiver configured to receive an FM signal from the other electrode device and convert it into a voltage signal;
an adjustment circuit configured to adjust the voltage signal from the FM receiver and provide it as an input to the amplifier circuit; and
a wireless transmitter configured to transmit biopotential information; and
a biosignal generation device configured to:
receive the biopotential information from the first and second electrode devices; and
generate a biosignal waveform based on the received biopotential information.

18. The biosignal measurement system of claim 17, wherein the FM transmitter of the first electrode device transmits at a first frequency and the FM transmitter of the second electrode device transmits at a second frequency different from the first frequency.

19. The biosignal measurement system of claim 17, wherein the FM transmitter and FM receiver of each electrode device are configured to use a human body as a communication channel for transmitting and receiving FM signals.

20. The biosignal measurement system of claim 17, wherein the FM transmitter comprises a voltage-controlled oscillator and the FM receiver comprises a phase-locked loop.

21. The biosignal measurement system of claim 20, wherein the adjustment circuit comprises an operational amplifier configured to adjust at least one of an amplification condition or an offset to match voltage-frequency characteristics between the voltage-controlled oscillator of one electrode device and the voltage-controlled oscillator of the phase-locked loop of the other electrode device.

22. The biosignal measurement system of claim 17, wherein the biosignal generation device is incorporated into one of the first or second electrode devices.

23. The biosignal measurement system of claim 17, wherein the first and second electrode devices are configured to be attached to any two positions of four limbs of the human body, and the biosignal generation device is configured to generate an electrocardiographic signal waveform based on the biopotential information from the first and second electrode devices.
